(19) United States
(12) Patent Application Publication
(10) Pub. No.: US 2015/0230976 A1
(43) Pub. Date: Aug. 20, 2015

(54) COOLING SYRINGE AND METHOD FOR INDUCING SELECTIVE HYPOTHERMIA OF A BODY TISSUE DURING MINIMALLY INVASIVE SURGERY

(71) Applicant: HENRY FORD HEALTH SYSTEM, Detroit,, MI (US)

(72) Inventors: Craig Glenn Rogers, Plymouth, MI (US); Wooju Jeong, Franklin, MI (US); Mani Menon, Bloomfield Hills, MI (US)

(21) Appl. No.: 14/430,995

(22) PCT Filed: Sep. 25, 2013
(86) PCT No.: PCT/US2013/061606

§ 371 Date: Mar. 25, 2015

Related U.S. Application Data
(60) Provisional application No. 61/705,615, filed on Sep. 25, 2012.

(51) Int. Cl. A61F 7/12; A61B 19/00; A61B 17/3205; A61F 7/00

(52) U.S. Cl. CPC A61F 7/12; A61F 7/0085; A61B 19/2203; A61B 17/3205; A61F 2007/0063; A61F 2007/126

(57) ABSTRACT

A cooling syringe and method are provided for inducing selective hypothermia of a body tissue during minimally invasive surgery. The cooling syringe includes an elongated, hollow housing configured to receive a cooling substance, the housing having a proximal end and a distal end. An actuator is at least partially received within the housing, the actuator including an elongated shaft having a proximal end and a distal end. The actuator includes a handle portion at the shaft proximal end and an end cap received on the shaft distal end, the shaft and end cap together forming a plunger. A guidance cap having a central channel is received on the shaft and disposed at a proximal end thereof. The actuator is movable with respect to the guidance cap and within the housing to dispense the cooling substance from the housing distal end.

### CROSS-REFERENCE TO RELATED APPLICATIONS

This application claims the benefit of U.S. provisional application Ser. No. 61/705,615 filed Sep. 25, 2012, the disclosure of which is hereby incorporated in its entirety by reference herein.

### TECHNICAL FIELD

Embodiments relate to a cooling syringe and method for inducing selective hypothermia of body tissues, such as the kidney and prostate, during minimally invasive surgery.

### BACKGROUND

According to the American Cancer Society, prostate cancer is the most common cancer in men, other than skin cancer, and the second-leading cause of cancer death among men. However, the death rate for prostate cancer is decreasing due to early detection and advancements in treatment. Minimally invasive surgical interventions, namely laparoscopic or robotic surgery, can be used to remove the cancerous prostate tissue with high success rates. However, outcomes may be further improved by selective hypothermia of the prostate during the surgical procedure. One of the drawbacks of robot-assisted radical prostatectomy (RARP) is the lack of tactile sensation and the ability to determine possible tumor margins.

Over 50,000 patients suffer from renal cell carcinoma annually in the United States and treatment often requires surgical removal of the cancerous tissue via a partial nephrectomy. During this surgery, the blood vessels to the kidney are usually clamped to allow the surgeon to remove the tumor and sew the kidney back together in a bloodless field. However, the loss of blood supply can damage the remaining normal kidney and every additional minute on clamp can cause more damage. Robotic partial nephrectomy (RPN) has become an alternative to open partial nephrectomy (OPN) for the treatment of small renal masses, but the removal of complex tumors may require a longer ischemic time. In open procedures, ice slush may be introduced around the kidney to create renal hypothermia, providing a broader ischemic window for complex excision and reconstruction. The ability to cool the kidney and grossly evaluate the excised tumor during open procedures has been difficult to replicate in a reliable fashion using minimally-invasive approaches.

### SUMMARY

In at least one embodiment, a cooling syringe is provided for inducing selective hypothermia of a body tissue during minimally invasive surgery. The cooling syringe includes an elongated, hollow housing configured to receive a cooling substance, the housing having a proximal end and a distal end. An actuator is at least partially received within the housing, the actuator including an elongated shaft having a proximal end and a distal end. The actuator includes a handle portion at the shaft proximal end and an end cap received on the shaft distal end, the shaft and end cap together forming a plunger. A guidance cap having a central channel is received on the shaft and disposed at a proximal end thereof. The actuator is movable with respect to the guidance cap and within the housing to dispense the cooling substance from the housing distal end.

In another embodiment, a method is provided for inducing selective hypothermia of a body tissue during minimally invasive surgery. The method includes providing a cooling syringe having an elongated, hollow housing configured to receive a cooling substance, the housing having a proximal end and a distal end, an actuator at least partially received within the housing, the actuator including an elongated shaft having a proximal end and a distal end, the actuator including a handle portion at the shaft proximal end and an end cap received on the shaft distal end, the shaft and end cap together forming a plunger, and a guidance cap having a central channel, the guidance cap received on the shaft and disposed at a proximal end thereof. The method further includes inserting the syringe through a surgical access port and into a body cavity such that the housing distal end is adjacent to the body tissue, and advancing the actuator toward the housing distal end to dispense the cooling substance from the housing distal end and directly onto the body tissue.

In another embodiment, a method is provided for inducing selective hypothermia of kidney tissue during a minimally invasive, robotic partial nephrectomy surgery. The method includes providing a cooling syringe having an elongated, hollow housing configured to receive a cooling substance, the housing having a proximal end and a distal end, and an actuator at least partially received within the housing, the actuator including an elongated shaft having a proximal end and a distal end, the actuator including a handle portion at the shaft proximal end and an end cap received on the shaft distal end, the shaft and end cap together forming a plunger. The method further includes inserting at least one robotic instrument through a surgical access port and into the abdominal cavity, and inserting the syringe through the surgical access port and into the abdominal cavity such that the housing distal end is adjacent to the kidney tissue. The method further includes clamping the renal artery, and advancing the actuator toward the housing distal end to dispense the cooling substance from the housing distal end and directly onto the kidney tissue.

### BRIEF DESCRIPTION OF THE DRAWINGS

FIG. 1 is a perspective view of a cooling syringe according to an embodiment;

FIG. 2 is a perspective view of actuator and housing components of the cooling syringe according to an embodiment;

FIG. 3 is a perspective view of an end cap of the cooling syringe according to one embodiment;

FIG. 4 is a perspective, exploded view of the shaft distal end and an end cap of the cooling syringe according to another embodiment;

FIG. 5 is a perspective view of a guidance cap of the cooling syringe according to an embodiment; and

FIG. 6 is a schematic representation of operation of the cooling syringe according to an embodiment.

### DETAILED DESCRIPTION

As required, detailed embodiments of the present invention are disclosed herein; however, it is to be understood that the disclosed embodiments are merely exemplary of the invention that may be embodied in various and alternative forms. The figures are not necessarily to scale; some features may be exaggerated or minimized to show details of particular components. Therefore, specific structural and functional details disclosed herein are not to be interpreted as limiting, but merely as a representative basis for teaching one skilled in the art to variously employ the present invention.

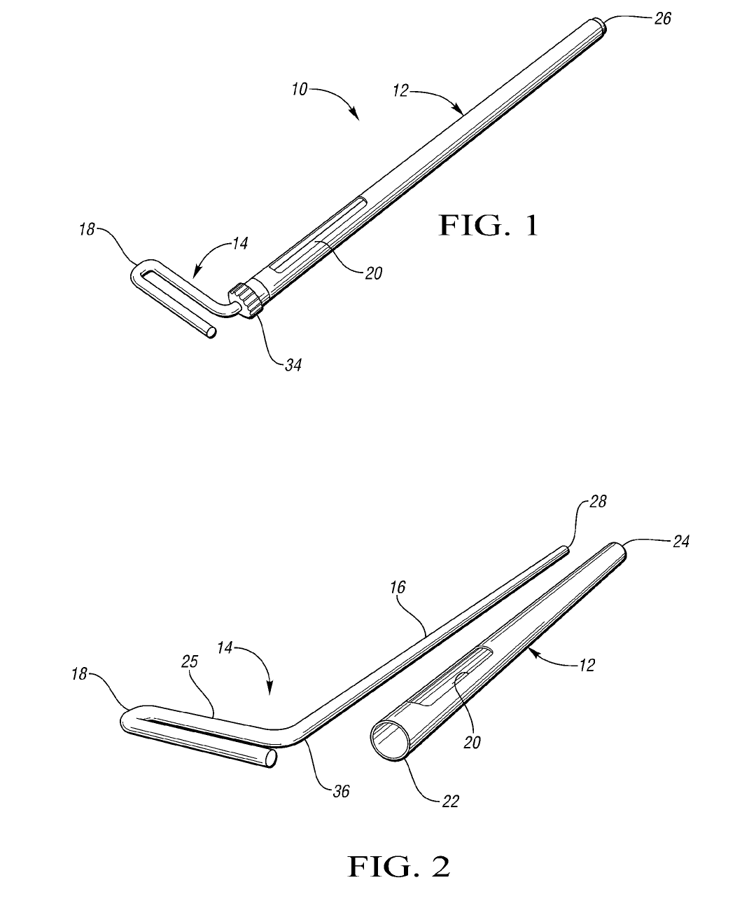

In accordance with an embodiment, a cooling syringe 10 is provided for delivering a cooling substance, such as ice slush, to a body tissue during minimally invasive surgery for inducing selective hypothermia. As illustrated in FIG. 1, in one embodiment the cooling syringe 10 comprises an elongated, generally cylindrical, hollow housing 12 and an actuator 14 at least partially received within the housing 12. As shown in FIG. 2, the actuator 14 includes an elongated shaft 16 and a handle portion 18. The housing 12 may include a window 20, such as adjacent a proximal end 22 thereof, for viewing the volume of ice slush within the housing 12 as well as the advancement of the shaft 16 within the housing 12. In one embodiment, a distal end 24 of the housing 12 is not tapered, as it would be in a typical syringe, and instead the housing 12 has a substantially uniform diameter along its length so as to allow ice slush to be easily backloaded into the syringe 10 and pushed out over the kidney, prostate, or other applicable tissue. In one embodiment, the handle portion 18 may be disposed with an axis generally perpendicular to a longitudinal axis of the shaft 16. The handle portion 18 may have any shape, such as the generally U-shaped configuration depicted herein, suitable for gripping and maneuvering of the actuator 14 by a user, and may include an applied grip material 25 or other grip surface or surface treatment to facilitate handling.

The components of the cooling syringe 10 may be constructed from a plastic material, and be sterile and disposable. In another embodiment, the syringe components may be constructed from a metallic material, such as stainless steel, so as to be sterilizable and reusable. The housing 12 and actuator 14 may be configured to be rigid or flexible. In one embodiment, the cooling syringe 10 has a capacity of about 150-200 cc. Housing 12 may have a length of about 50-60 cm, and window 20 may have a length of about 10-15 cm. Shaft 16 may have a length of about 50-60 cm and a diameter of about 8-12 mm, and handle portion 18 may have a width of about 8-12 cm. It is understood that these capacities and dimensions are merely exemplary, and other specifications of syringe 10 are also fully contemplated.

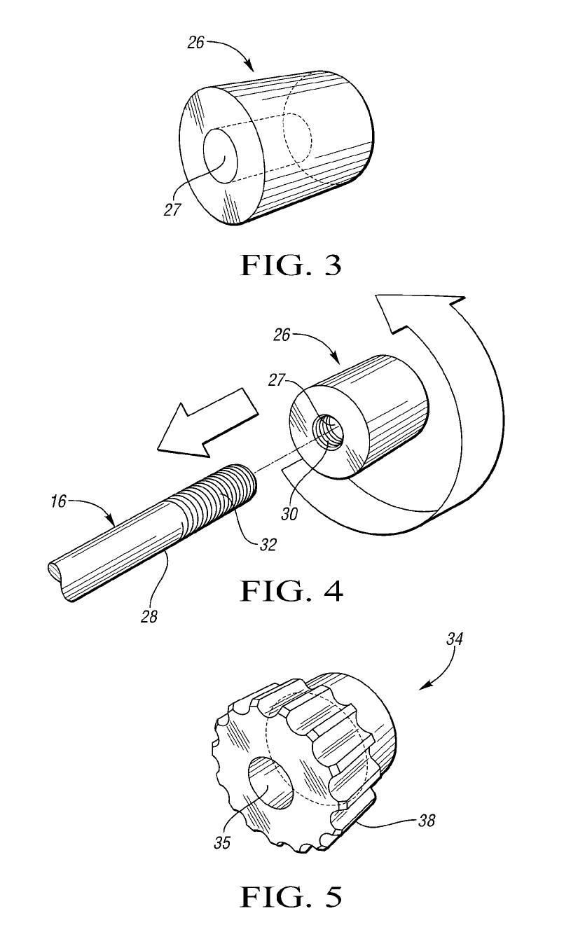

FIG. 3 depicts an end cap 26 which is disposed on a distal end 28 of the shaft 16, thereby creating a plunger. The end cap 26 may be secured on the shaft distal end 28 via an interference fit with a cavity 27 formed therein, or alternatively may include internal threads 30 with the cavity 27 as shown in FIG. 4 which mate with threads 32 on the shaft distal end 28. An embodiment of a guidance cap 34 having a central channel 35 and received on shaft 16 at a proximal end 36 thereof is illustrated in FIG. 5. In one embodiment, the guidance cap 34 may be coupled to the housing proximal end 22. The central channel 35 may serve to guide and stabilize the shaft 16 as it is advanced into or withdrawn from the housing 12. Guidance cap 34 may include a scalloped circumferential edge 38 or other grip portion for facilitating a stable grip by a user.

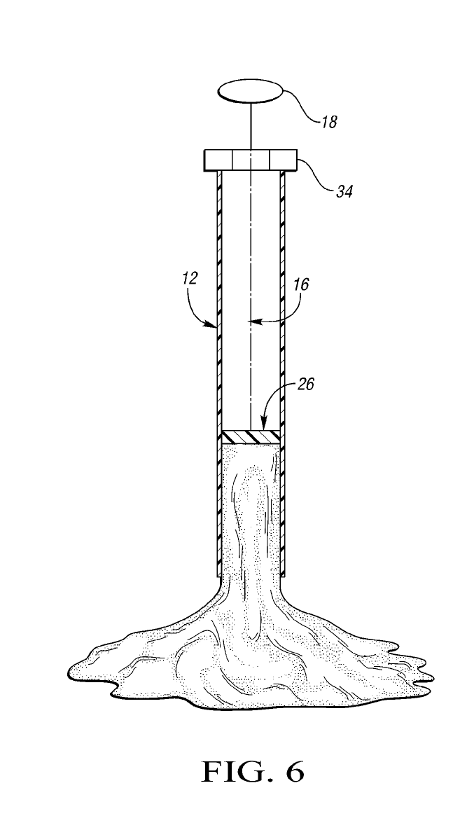

With reference to FIG. 6, in operation, actuator 14 can be withdrawn toward the proximal end 22 of housing 12 so that housing 12 can be filled with ice slush. Advancing the plunger (shaft 16 and end cap 26) within the housing 12, such as with a user gripping the handle portion 18 with one hand and stabilizing the syringe 10 by gripping the guidance cap 34 or housing 12 with the other hand, dispenses ice slush out of housing distal end 24. The user has the ability to control the flow rate of slush by the speed of advancement of the actuator 14 with respect to the housing 12.

The cooling syringe 10 may be inserted into the body cavity through an access hole associated with a laparoscopic surgical port, such as a GELPOINT® access port (Applied Medical, CA, USA) placed near the umbilicus. The elongated housing 12 and shaft 16 allow the end of the cooling syringe 10 to be advanced over the kidney or other tissue so that ice slush can be applied directly onto the surface of the tissue. The syringe 10 may advanced through a trocar and may be guided by endoscope viewing for depositing ice slush around the tissue to induce protective hypothermia. As the ice slush melts, the water can be suctioned away and additional ice slush can be applied as needed. Multiple cooling syringes 10 could be used in a single case, allowing several syringes 10 to be preloaded with ice slush to allow for multiple applications during the case without having to reload the syringe, such as while on clamp. Other methods of pushing the ice slush through the syringe 10 could also be used, such as a trigger mechanism (like a caulking gun) or a plunger having a screw that is twisted.

In one study, RARP was modified using a GELPOINT® access port to allow immediate retrieval of the prostate for on-table examination and targeted frozen section biopsies (MORE procedure). The aim of this technique was to reduce the positive surgical margin (PSM) rate in patients with high-risk disease.

Sixty-two patients with a probability of extracapsular extension (EPE) greater than 25% were selected to undergo MORE-RARP. MORE include a GELPOINT® placed in the periumbilical region with a 12 mm camera and 5 mm retrieval ports placed. Following excision of the prostate, the prostate was retrieved through the GELPOINT®, and examined on-table by the surgeon. Lesions suspicious for positive margins were sent for frozen biopsy while node dissection was performed. Biopsies suggestive of cancer resulted in more tissue being removed in order to achieve negative margins.

The MORE procedure included introducing ice slush into the pelvic cavity through the GELPOINT® using open-ended cooling syringes. Patients undergoing MORE were compared to a control group of 30 consecutive patients with pT₃ disease undergoing RARP. The results for positive surgical margins were MORE (16%) and control (46.7%), and the results for negative surgical margins were MORE (84%) and control (53.3%). The overall positive predictive value for extraprostatic extension of the prostate cancer was 15/25 (60%). The MORE technique reduced the positive surgical margin rate in RARP in pathological T₃ disease. Frozen sites according to surgeon's tactile sensations had 60% of positive predictive value for extraprostatic extension of the prostate cancer.

In another study, six patients underwent a modification to a standard technique of RPN called Robotic Intracorporeal Cooling and Extraction (R-ICE). A transperitoneal approach was used in four patients and two underwent a retroperitoneal approach. Patient demographics are provided in Table 1.

TABLE 1
Demographics and preoperative data on
patients undergoing RPN with iced ischemia

| | |
|---|---|
| No. of patients | 6 |
| Median age (range) | 61 (48-69) |
| Side (right/left) | 5/1 |
| Mean body mass index (kg/m2) | 34.5 (29.7-39.9) |
| (range) | |
| Mean serum creatinine (mg/dl) | 0.94 (0.8-1.3) |
| (range) | |
| Mean GFR (ml/min/1.73 m²) | 87.6 (59-102) |
| No. of patients with prior | 3 |
| abdominal surgery | |
| Indication for partial | Solitary kidney (1) |
| nephrectomy (n): | Bilateral masses (1) |
| | Chronic renal insufficiency (1 or 2) |
| Mean cm tumor size (range) | 2.7 (1.7-3.8) |
| No. tumor location (n): | Upper pole (2) |
| | Inter-polar (2) |
| | Lower pole (1) [missing data 1] |
| Median nephrometry score (range) | 8 (6-10) |

A GELPOINT® access port was used during RPN to permit placement of iced saline slush into the abdominal cavity and rapid tumor extraction. The GELPOINT® was inserted through an incision at the paramedian line or below the twelfth rib, for transperitoneal and retroperitoneal approaches, respectively. All patients were placed in the standard flank position. In the retroperitoneal approach, a 2-cm transverse skin incision was made below the tip of the 12th rib. The muscles were split bluntly to expose the thoracolumbar fascia which was incised to enter the retroperitoneum. The retroperitoneal space was developed with blunt finger dissection and balloon dilation (U.S. Surgical, Norwalk, Conn.) under direct laparoscopic vision. The skin incision was extended to accommodate placement of the GELPOINT® wound retractor. The camera and assistant ports were preplaced through the GELPOINT® maximizing separation, and the GELPOINT® and trocars were attached to the wound retractor. Robotic trocars were placed on each side of the GELPOINT® with an additional medial robotic trocar for the third robotic arm to provide lift on the kidney. In the transperitoneal approach, a paramedian incision was made lateral to the rectus muscle and the GELPOINT® placed after gaining open entry to the peritoneum. A grasper for liver retraction in right-sided cases was placed directly through the GELPOINT® without the need for a port.

Robotic instruments used included a fenestrated bipolar grasper in the non-dominant hand and monopolar curved scissors in the dominant hand, which was exchanged for a robotic needle-driver for renorrhaphy. Intraoperative ultrasound for tumor delineation was performed using a robotic ultrasound probe (Hitachi-Aloka, Tokyo, Japan) and clamping of the renal artery was performed using robotic bulldog clamps (Scanlan Inc.). For both approaches, the kidney was mobilized and the renal vessels were dissected. The renal artery was encircled with a vessel loop to facilitate clamping and identification of the renal hilum after ice slush delivery.

Sterile iced saline slush was created in a slush machine (Hush Slush® system, OR solutions, Ecolab Inc., MN, USA). Disposable syringes (30 cc, 70 cc) were modified by cutting off the nozzle end to make a wider opening and were prefilled with ice slush in preparation for rapid injection. Following clamping of the renal artery, syringes were placed through the GELPOINT® at the site of the removed assistant port allowing injection of iced saline slush around the kidney. Repeated rapid injections of ice slush were performed to cover the kidney surface. Renal parenchymal and core body temperatures were measured by thermal probes placed in the esophagus and kidney, respectively. Upper and lower body warmers were used to maintain core body temperature. In total, approximately 400-600 cc of ice was delivered in all patients.

Tumor excision was performed immediately after ice delivery. Ice could be reapplied as needed while on clamp. Following tumor excision, the tumor was placed in a 10 mm retrieval bag (Endocatch, Ethicon Endosurgery, Norwalk, Conn., USA) and immediately extracted through the GELPOINT®. This allowed the whole tumor to be examined on-table intra-operatively for gross margin assessment during renorrhaphy.

An early unclamping technique was employed with removal of the arterial clamp after suturing the inner layer of the renorrhaphy. Renorrhaphy was completed with outer interrupted capsular sutures followed by application of hemostatic agents. Excess ice slush could be removed with suction and irrigation. Peri-operative and post-operative data are provided in Table 2. There were no post-operative complications. Intra-operative assessment of the excised tumor showed adequate gross margins in all cases.

TABLE 2
Intra-operative and post-operative outcomes

| | |
|---|---|
| Blood loss (ml) (range) | 338 (100-1000) |
| Operative time (mins) (range) | 274 (235-366) |
| Mean cold ischemia time (mins) (range) | 20 (8-37) |
| Pathological tumor size (cm) (range) | 2.9 (1.4-5.2) |
| Mean serum creatinine (mg/dl) (range) | — |
| Mean GFR (ml/min/1.73 m²) | — |
| Pathology | Papillary RCC (2) |
| | Clear cell RCC (2) |
| | Oncocytoma (1) |
| | missing data [1] |
| Complications | Intra-operative bleeding |
| | requiring transfusion |
| Median length of stay (days) (range) | 2 (2-4) |
| F/U (months) | — |

We had previously tested the ability to achieve adequate parenchymal temperatures with our ice slush cooling technique on a porcine model using thermal needle probes to monitor renal parenchymal temperatures. In the three test cases, the kidneys were cooled to a temperature <20° C. within 10 minutes and maintained the target temperature for the duration of hilar clamping. We then monitored renal parenchymal temperatures in a transperitoneal study patient, achieving parenchymal temperatures of <16° C. within seven minutes of cold ischemia. There were no significant decreases in core body temperature (<0.5° C.) during the procedure.

Despite the increase in utilization of partial nephrectomies (PN) over the last decade, the vast majority of PN's today are still performed using an open approach. In the U.S. Nationwide Inpatient Sample for 2008, laparoscopic partial nephrectomies (LPN) consisted of only 11.7% of partial nephrectomy procedures. One of the barriers to the broader uptake of minimally-invasive PN may be the concern that the operation cannot safely be done within reasonable time constraints of warm ischemia. Renal hypothermia may be necessary for prolonged warm ischemia times, particularly in patients with renal insufficiency. Renal hypothermia lowers the metabolic rate and protects the kidney from ischemic damage, permitting longer periods of ischemia to accomplish PN. At present there is no standardized method for inducing renal hypothermia during minimally invasive PN. Easy, reliable and reproducible cold ischemia during minimally invasive PN remains an elusive puzzle that many investigators have tried to solve.

Although previous methods of renal hypothermia for LPN have demonstrated effective parenchymal cooling, most techniques require specific equipment or expertise and are too complex or impractical for routine use. Transarterial cold perfusion requires an interventional radiologist and predisposes the patient to risk of vascular injury or thrombosis. Alternative methods such as retrograde ureteral cooling involve retrograde catheter placement and need for a post-operative stent. Also, incisions in the collecting system during this technique can compromise the degree of hypothermia and impair visualization. Irrigation with cold saline can lead to large amounts of fluid overflow into the peritoneum and could cause drops in the core temperature. The gold standard for inducing renal hypothermia is surface cooling with crushed ice as this achieves significantly lower cortical temperatures. However, standard ice slush for renal hypothermia during OPN is composed of dendritic ice crystals that do not flow well through small caliber laparoscopic cannulae.

With the disclosed R-ICE technique, we have demonstrated a modification to RPN that allows easy and reproducible introduction of iced saline slush to the kidney surface using the same slush machine required for OPN. Ice slush can be injected on to the kidney surface via a surgical access port without losing insufflation or extending an incision. A further advantage is the ability to immediately retrieve the excised specimen through the surgical port for gross margin assessment by the surgeon while the surgery is in progress. If gross inspection of the early extracted tumor were to raise questions of inadequate resection, additional resection or biopsies could be performed before the operation is over. The R-ICE technique offers advantages typically reserved to OPN, namely the ability to ice the kidney and evaluate the resected specimen.

The feasibility of ice slush renal hypothermia for retroperitoneal LPN has been demonstrated herein. The disclosed technique of R-ICE incorporating a surgical port such as the GELPOINT® can be used for both transperitoneal and retroperitoneal approaches. The GELPOINT® platform maintains insufflation while being a good conduit for the delivery of ice and specimen retrieval. Although the GELPOINT® has been used during retroperitoneal laparoscopic nephrectomy and LPN, it has not yet been reported in conjunction with the da Vinci robot for retroperitoneal RPN or with ice slush hypothermia with early tumor extraction.

Other advantages of using a surgical port such as the GELPOINT® in this setting is that patients with prior abdominal surgery and adhesions pose less of a difficulty, as open Hasson dissection with limited open adhesiolysis is feasible prior to GELPOINT® insertion. This was particularly helpful in patient 6 in our series. The retroperitoneal approach for R-ICE RPN facilitates management of posterior lesions and patients who have undergone extensive intra-abdominal surgery. We found the retroperitoneal approach to be more ideal for containing ice around the kidney compared to the transperitoneal route.

In summary, the R-ICE RPN technique allows for ice delivery and early specimen evaluation during RPN with acceptable operative times and short-term outcomes. Iced cold ischemia during RPN is a promising new technique that has the potential to expand the limitations of RPN.

The cooling syringe described herein could cause a paradigm shift in the way that prostatectomy and partial nephrectomy surgeries are performed so that tissue cooling could be routinely offered for all minimally invasive cases. The indication for open prostatectomy and partial nephrectomy would decrease as most of these could now be done through a minimally invasive approach. Other accessory technologies could be used in conjunction with the cooling syringe to facilitate tissue cooling. For example, small bags of ice could be placed through the surgical port underneath or around the kidney or prostate. A specialized sponge could be placed, for example, around the base of the kidney for both cooling and to minimize spillage of melted ice. The surgical port could also be modified to optimize cooling syringe and ice bag insertion.

While exemplary embodiments are described above, it is not intended that these embodiments describe all possible forms of the invention. Rather, the words used in the specification are words of description rather than limitation, and it is understood that various changes may be made without departing from the spirit and scope of the invention. Additionally, the features of various implementing embodiments may be combined to form further embodiments of the invention.

What is claimed is:

1. A cooling syringe for inducing selective hypothermia of a body tissue during minimally invasive surgery, comprising:
an elongated, hollow housing configured to receive a cooling substance, the housing having a proximal end and a distal end;
an actuator at least partially received within the housing, the actuator including an elongated shaft having a proximal end and a distal end, the actuator including a handle portion at the shaft proximal end and an end cap received on the shaft distal end, the shaft and end cap together forming a plunger; and
a guidance cap having a central channel, the guidance cap received on the shaft and disposed at a proximal end thereof,
wherein the actuator is movable with respect to the guidance cap and within the housing to dispense the cooling substance from the housing distal end.

2. The cooling syringe of claim 1, wherein the housing includes a window adjacent the housing proximal end.

3. The cooling syringe of claim 1, wherein the housing has a substantially uniform diameter along its length.

4. The cooling syringe of claim 1, wherein a circumferential edge of the guidance cap includes a grip portion.

5. The cooling syringe of claim 1, wherein the end cap includes a cavity into which the shaft distal end is received via an interference fit.

6. The cooling syringe of claim 1, wherein the end cap includes a cavity having an internal threaded surface which mates with threads formed on the shaft distal end for securing the end cap to the shaft.

7. The cooling syringe of claim 1, wherein the handle is generally U-shaped.

8. The cooling syringe of claim 1, wherein the handle is disposed along an axis generally perpendicular to a longitudinal axis of the shaft.

9. The cooling syringe of claim 1, wherein the handle includes a grip surface.

10. The cooling syringe of claim 1, wherein the housing has a capacity of about 150-200 cc of the cooling substance.

11. The cooling syringe of claim 1, wherein a length of the housing and a length of the shaft are each about 50-60 cm.

12. A method for inducing selective hypothermia of a body tissue during minimally invasive surgery, comprising:
providing a cooling syringe having an elongated, hollow housing configured to receive a cooling substance, the housing having a proximal end and a distal end, an actuator at least partially received within the housing, the actuator including an elongated shaft having a proximal end and a distal end, the actuator including a handle portion at the shaft proximal end and an end cap received on the shaft distal end, the shaft and end cap together forming a plunger, and a guidance cap having a central channel, the guidance cap received on the shaft and disposed at a proximal end thereof;
inserting the syringe through a surgical access port and into a body cavity such that the housing distal end is adjacent to the body tissue; and
advancing the actuator toward the housing distal end to dispense the cooling substance from the housing distal end and directly onto the body tissue.

13. The method of claim 12, further comprising guiding insertion of the syringe with an endoscope.

14. The method of claim 12, further comprising suctioning excess cooling substance from the body cavity.

15. A method for inducing selective hypothermia of kidney tissue during a minimally invasive, robotic partial nephrectomy surgery, comprising:
providing a cooling syringe having an elongated, hollow housing configured to receive a cooling substance, the housing having a proximal end and a distal end, and an actuator at least partially received within the housing, the actuator including an elongated shaft having a proximal end and a distal end, the actuator including a handle portion at the shaft proximal end and an end cap received on the shaft distal end, the shaft and end cap together forming a plunger;
inserting at least one robotic instrument through a surgical access port and into the abdominal cavity;
inserting the syringe through the surgical access port and into the abdominal cavity such that the housing distal end is adjacent to the kidney tissue;
clamping the renal artery; and
advancing the actuator toward the housing distal end to dispense the cooling substance from the housing distal end and directly onto the kidney tissue.

16. The method of claim 15, further comprising excising tumor-containing kidney tissue through the surgical access port for evaluation while the renal artery remains clamped during the surgery.

17. The method of claim 15, further comprising suctioning excess cooling substance from the abdominal cavity.

18. The method of claim 15, wherein dispensing the cooling substance includes delivering about 400-600 cc of the cooling substance to the kidney tissue.

19. The method of claim 15, wherein the robotic partial nephrectomy uses a retroperitoneal approach.

20. The method of claim 15, wherein the robotic partial nephrectomy uses a transperitoneal approach.
